(19) United States
(12) Patent Application Publication
(10) Pub. No.: US 2022/0036994 A1
(43) Pub. Date: Feb. 3, 2022

(54) PRESENTATION SYSTEM, PRESENTATION METHOD, AND PROGRAM

(71) Applicant: Toyota Jidosha Kabushiki Kaisha, Toyota-shi Aichi-ken (JP)

(72) Inventors: Toru Miyagawa, Seto-shi (JP); Issei Nakashima, Toyota-shi (JP); Makoto Kobayashi, Nisshin-shi (JP); Takuya Iwata, Toyota-shi (JP); Hisataka Yuasa, Nagoya-shi (JP); Masayuki Imaida, Ichinomiya-shi (JP); Keisuke Suga, Nagoya-shi (JP); Eiichi Saitoh, Nagoya-shi (JP); Yohei Otaka, Kariya-shi (JP); Satoshi Hirano, Nagoya-shi (JP)

(73) Assignee: Toyota Jidosha Kabushiki Kaisha, Toyota-shi Aichi-ken (JP)

(21) Appl. No.: 17/385,090

(22) Filed: Jul. 26, 2021

(30) Foreign Application Priority Data
Jul. 28, 2020 (JP) 2020-127547

(51) Int. Cl. G16H 20/30; G06F 30/13

(52) U.S. Cl. CPC G16H 20/30; G06F 30/13

(57) ABSTRACT

A presentation system includes a storage unit, an acquisition unit, a determination unit, and an output unit. The storage unit is configured to store definition information defining a housing environment according to a physical ability. The acquisition unit is configured to acquire first physical ability information of a trainee who performs training for the purpose of maintaining or recovering the physical ability before the training, and plan information which is a plan of the training. The determination unit is configured to estimate second physical ability information which is the physical ability of the trainee after an end of the plan based on the first physical ability information and the plan information, and determine the housing environment corresponding to the second physical ability information based on the definition information. The output unit is configured to output presentation information including the determined housing environment.

### CROSS REFERENCE TO RELATED APPLICATIONS

This application is based upon and claims the benefit of priority from Japanese patent application No. 2020-127547, filed on Jul. 28, 2020, the disclosure of which is incorporated herein in its entirety by reference.

### BACKGROUND

The present disclosure relates to a presentation system, presentation method, and a program.

A training system for rehabilitation (rehabilitation and training) for the purpose of functional recovery of a physical action of a trainee has been developed. For example, a walking training apparatus disclosed in Japanese Unexamined Patent Application Publication No. 2015-223294 includes a walking assistance apparatus, first pulling means, and second pulling means. The walking assistance apparatus is attached to the trainee's leg and assists the trainee in walking. The first pulling means pulls at least one of the walking assistance apparatus and the trainee's leg upward and forward. The second pulling means pulls at least one of the walking assistance apparatus and the trainee's leg upward and backward.

### SUMMARY

It should be noted that a patient (i.e., a trainee) under rehabilitation (hereinafter also referred to as a rehabilitation patient (or a rehabilitation trainee)) resumes his/her life in his/her house after performing rehabilitation at a rehabilitation facility. At this time, the trainee may have difficulty in his/her ordinary life in the house. Therefore, rehabilitation patients often renovate their houses to adjust to their physical actions. It is desirable that such renovation be completed when the rehabilitation patient returns to his/her house. However, the degrees of recovery of rehabilitation patients and their housing environments widely vary from one patient to another. Therefore, it is difficult to appropriately predict the degree of recovery of a rehabilitation patient and determine the housing environment and the like corresponding to the predicted degree of recovery. If it is not possible to prepare the housing environment appropriately, the burden is imposed on the trainee who lives in his/her house.

The present disclosure has been made in order to solve such a problem and an object thereof is to provide a presentation system and the like which prevent or minimize an increase in a burden of a trainee.

A presentation system according to the present disclosure includes a storage unit, an acquisition unit, a determination unit, and an output unit. The storage unit is configured to store definition information defining a housing environment according to a physical ability. The acquisition unit is configured to acquire first physical ability information of a trainee who performs training for the purpose of maintaining or recovering the physical ability before the training, and plan information which is a plan of the training. The determination unit is configured to estimate second physical ability information which is the physical ability of the trainee after an end of the plan based on the first physical ability information and the plan information, and determine the housing environment corresponding to the second physical ability information based on the definition information. The output unit is configured to output presentation information including the determined housing environment.

With such a configuration, the presentation system can present a housing environment suitable for the user.

In the above presentation system, the acquisition unit may be further configured to acquire profile information of the trainee, and the determination unit may be configured to estimate the second physical ability information which is the physical ability of the trainee after the end of the plan based on the first physical ability information, the plan information, and the profile information. Thus, the presentation system can improve the accuracy of the estimation of the second physical ability information.

In the above presentation system, the storage unit may be further configured to store statistical information related to the training performed by a plurality of other trainees, and the determination unit may be configured to estimate the second physical ability information which is the physical ability of the trainee after the end of the plan based on the first physical ability information, the plan information, and the statistical information. Thus, the presentation system can improve the accuracy of the estimation of the second physical ability information.

In the above presentation system, the storage unit may be configured to further store preparation information related to a preparation period for preparing the housing environment, the determination unit may be configured to determine that a notification concerning a start of preparation of the housing environment is to be issued at a time a preparation period before an end of the plan, the preparation period being for preparing the housing environment included in the presentation information, and the output unit may be configured to output the notification. Thus, the presentation system can output the presentation information at a suitable timing.

In the above presentation system, the storage unit may be configured to store the presentation information, the acquisition unit may be configured to acquire progress information regarding the training plan, and the determination unit may be configured to update the second physical ability information according to the progress information and update the housing environment according to the updated second physical ability information. Thus, the presentation system can flexibly present housing environment information.

In the above presentation system, the storage unit may be configured to store the presentation information, and when the plan information is updated, the determination unit may be configured to update the second physical ability information according to the updated plan information, and update the housing environment according to the updated second physical ability information. Thus, the presentation system can flexibly present the housing environment information.

In the above presentation system, the storage unit may be further configured to store provider information related to a provider of the housing environment, and the output unit may be configured to output the presentation information including the provider information related to the determined housing environment. Thus, the presentation system can output presentation information of more suitable contents.

The above presentation system may further include an instruction reception unit configured to receive an instruction for selecting the provider, and a transmission unit configured to transmit selection information related to the selection to the provider related to the selection received by the instruction reception unit. Thus, the presentation system can further reduce a burden on the trainee.

A presentation method according to the present disclosure includes steps of storing, acquiring, determining, and outputting. In the step of storing, definition information defining a housing environment according to a physical ability is stored. In the step of acquiring, first physical ability information of a trainee who performs training for the purpose of maintaining or recovering the physical ability before the training, and plan information which is a plan of the training are acquired. In the step of determining, second physical ability information which is the physical ability of the trainee after an end of the plan is estimated based on the first physical ability information and the plan information, and the housing environment corresponding to the second physical ability information based on the definition information is determined. In the step of outputting, presentation information including the determined housing environment is output.

With such a configuration, the presentation system can present a housing environment suitable for the user.

A program according to the present disclosure causes a computer to execute the following presentation method. The presentation method includes steps of storing, acquiring, determining, and outputting. In the step of storing, definition information defining a housing environment according to a physical ability is stored. In the step of acquiring, first physical ability information of a trainee who performs training for the purpose of maintaining or recovering the physical ability before the training, and plan information which is a plan of the training are acquired. In the step of determining, second physical ability information which is the physical ability of the trainee after an end of the plan is estimated based on the first physical ability information and the plan information, and the housing environment corresponding to the second physical ability information based on the definition information is determined. In the step of outputting, presentation information including the determined housing environment is output.

With such a configuration, the presentation system can present a housing environment suitable for the user.

According to the present disclosure, it is possible to provide a presentation system and the like which prevent or minimize an increase in a burden of a trainee.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing an overall configuration of a rehabilitation system according to embodiments;

FIG. 2 is a block diagram of a presentation system according to the first embodiment;

FIG. 3 is a flowchart of a presentation system according to the first embodiment;

FIG. 4 shows a first example of definition information according to the first embodiment;

FIG. 5 shows a second example of the definition information according to the first embodiment;

FIG. 6 shows statistical information according to the first embodiment;

FIG. 7 shows an example of information acquired by an acquisition unit according to the first embodiment;

FIG. 8 shows an example of presentation information according to the first embodiment;

FIG. 9 is a block diagram of a presentation system according to a second embodiment;

FIG. 10 is a flowchart of the presentation system according to the second embodiment;

FIG. 11 is a timing chart of the presentation system according to the second embodiment;

FIG. 12 is a block diagram of a presentation system according to a third embodiment; and

FIG. 13 is a flowchart of the presentation system according to the third embodiment.

### DESCRIPTION OF EMBODIMENTS

Hereinafter, the present disclosure will be described through embodiments of the disclosure, but the disclosure according to the claims is not limited to the following embodiments. Further, not all of the configurations described in the embodiments are essential as means for solving the problem. For clarity of description, the following descriptions and drawings have been omitted and simplified as appropriate. In the drawings, the same elements are denoted by the same reference signs, and repeated description is omitted if necessary.

### First Embodiment

Embodiments of the present disclosure will be described below with reference to the drawings. With a rehabilitation system according to this embodiment, a trainee performs a plurality of types of training for the purpose of maintaining or recovering his/her physical ability of the trainee, and the rehabilitation system according to this embodiment assists or supports Activities of Daily Living (ADL).

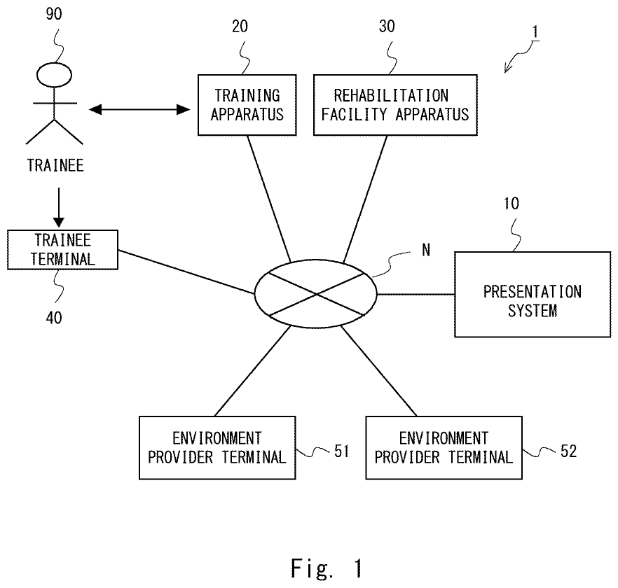

FIG. 1 is a block diagram showing an overall configuration of a rehabilitation system according to a first embodiment. A rehabilitation system 1 includes a training apparatus 20 used by a trainee 90, a rehabilitation facility apparatus 30, a trainee terminal 40, a presentation system 10 (also referred to as a presentation apparatus), an environment provider terminal 51, and an environment provider terminal 52. The training apparatus 20, the rehabilitation facility apparatus 30, the trainee terminal 40, the presentation system 10, the environment provider terminal 51, and the environment provider terminal 52 are connected to each other through a network N. Note that the network N is a communication line network such as the Internet, an intranet, a cellular phone network, and a LAN (Local Area Network).

The trainee 90 is a person who performs training in order to restore or maintain his/her own physical ability by using the training apparatus 20 or the like in a rehabilitation facility such as a medical institution. The trainee 90 is, for example, a rehabilitation patient who performs rehabilitation in order to restore his/her physical ability after surgery performed due to a serious illness or a serious injury. Alternatively, the trainee 90 is a person who performs rehabilitation to try to maintain his/her physical ability, such as an elderly person. Note that the term “rehabilitation” refers to a training process that is performed in order to restore or maintain a physical function or alleviate physical disability, irrespective of whether a health insurance is applied or not and whether a medical apparatus is used or not.

The training apparatus 20 is an apparatus by which the trainee 90 performs training in order to restore or maintain his/her physical ability in accordance with the assistance given by a training staff such as an instruction or help. As the training apparatus 20, for example, a rehabilitation support apparatus such as a walking training apparatus disclosed in Japanese Unexamined Patent Application Publication No. 2015-223294 can be used, though the training apparatus 20 is not limited to such rehabilitation support apparatuses.

In this case, the training apparatus 20 acquires detection data detected (measured) by internal or external sensors or the like during rehabilitation performed by the trainee 90. Note that the detection data is an example of the physical ability information and may be a set of measured values each of which corresponds to a respective one of a plurality of indexes (e.g., a plurality of sensors) for the measurement. Further, the detection data is data in which a measured value is associated with a measurement time and/or an index.

A typical example of the detection data is sensor data. The sensor data is sensor values detected by various sensors (not shown) of the training apparatus 20. For example, the sensor data includes an inclination angle of the trunk detected by the posture sensor, a load and an inclination angle detected by the handrail sensor, an angle detected by the angle sensor, etc. The sensors that output the sensor data are an acceleration sensor, an angular-velocity sensor, a position sensor, an optical sensor, a torque sensor, a weight sensor, etc. Further, encoders provided in motors of the winding mechanisms and the like included in the training apparatus 20 may be used as sensors. Further, a torque sensor (a load cell) of the motor may be used as a sensor, or a current detection unit that detects a driving current value for driving the motor may be used as a sensor.

Further, the sensor data may include, for example, line-of-sight data acquired by a line-of-sight detection sensor that detects a line of sight. Similar line-of-sight data can be obtained by detecting a line of sight of the trainee 90 by performing image processing based on an image taken by shooting at least an area including the eyes of the trainee 90, or obtained by determining the orientation (upward/downward etc.) of the face of the trainee 90 based on an image taken by shooting at least the face. Such data may also be included in the aforementioned detection data. Further, the detection data may be audio data (voice data) acquired by a voice acquisition unit, such as a microphone, that acquires a voice of the trainee 90 or the training staff, text data obtained by performing a voice analysis on the voice data, or data obtained by analyzing the text data. The voice of the training staff may include an encouraging talk to the trainee 90 about, for example, how to correct his/her walking. Further, the sensor data may be data obtained by detecting brain waves of the trainee 90 by using an electroencephalograph, or may be data obtained by detecting brain waves of the training staff by using an electroencephalograph.

Further, the line-of-sight detection sensor, a shooting unit that takes the above-described image, a microphone, and the like can be disposed in the training apparatus 20 itself. Alternatively, they can also be disposed in, for example, an eyeglass-type wearable terminal that is worn by the trainee 90. This terminal may include a wireless communication unit that wirelessly transmits and receives data by a wireless communication technique such as Bluetooth (Registered Trademark). Further, the training apparatus 20 may also include a wireless communication unit. In this way, the training apparatus 20 can acquire data acquired by the wearable terminal through wireless communication. Although the electroencephalograph is limited to those having high detection accuracy, it may be disposed in the training apparatus 20 itself and configured so that the electroencephalogram of the trainee 90 and that of the training staff can be separately detected. However, the electroencephalograph may be disposed at a position near the person whose brain waves are detected, such as being disposed in the above-described eyeglass-type wearable terminal (e.g., in a side frame of the eyeglasses).

Further, the detection unit that acquires detection data, such as sensors, is not limited to those described above. For example, the trainee 90 may wear clothes equipped with a wearable biosensor and/or a wearable touch sensor. Here, the clothes are not limited to those worn on the upper body. That is, they may be those worn on the lower body, a top-and-bottom set, or those attached to a part of the harness or the like. Further, a wireless communication unit like the one described above is provided in each of the clothes and the training apparatus 20. In this way, the training apparatus 20 can acquire data acquired by the wearable biological sensor or the wearable touch sensor through wireless communication. The wearable biosensor can acquire vital data such as the heart rate of the wearer. The wearable touch sensor can acquire data indicating information about a touch on the trainee 90, who is the wearer, made from the outside. That is, the wearable touch sensor can acquire data indicating information about a position where the training staff touched the trainee 90.

Further, the detection data is not limited to the values indicated by the detection signals detected by various sensors and the like. That is, they may include values calculated based on the detection signals from a plurality of sensors and statistical values obtained by statistically processing detection signals from one or a plurality of sensors or the like. As the statistical values, various statistical values such as an average value, a maximum value, a minimum value, and a standard deviation value may be used. Alternatively, they may be static statistical values or dynamic statistical values over a certain period such as one day, one training practice, or one walking cycle.

For example, the sensor data may include an open angle of the knee joint calculated from the angle between the upper-leg frame and the lower-leg frame detected by the angle sensor. Further, the sensor data of the angle sensor may include an angular velocity that is obtained by differentiate the angle. The sensor data of the acceleration sensor may be a velocity that is obtained by integrating the acceleration or a position that is obtained by integrating the acceleration twice.

As described above, data supplied directly or indirectly from the detection unit such as various sensors can be included in the detection data. Further, time information such as date and time at which the detection is performed or timing information other than the time can be added to the above-described detection data.

Note that the above-described detection data is merely an example and other detection data may be used. Further, some of the above-described detection data may not be used.

Note that the physical ability information may include index data about rehabilitation performed by the trainee 90 by using the training apparatus 20, including at least one of a symptom, a physical ability, and a degree of recovery of the trainee 90. In other words, the physical ability information may include symptom information, a Br. stage, a SIAS, an initial walking FIM, a latest walking FIM, and the like of the trainee 90. Note that the physical ability information may include various data indicating the physical ability of the trainee 90.

The physical ability information may include an action level described later. The action level is an index indicating a level of a physical action set using at least information related to the type of a tool used by the trainee in the physical action and an achievement degree of the physical action.

Further, the training apparatus 20 may associate trainee attribute information such as an attribute and physical information of the trainee 90 with the above-described physical ability information and store them in an internal storage device. Note that the trainee attribute information may include an age, a gender, a physique (a height, a weight, etc.), and the like of the trainee 90.

The training apparatus 20 may transmit the physical ability information and the like to the rehabilitation facility apparatus 30 through the network N and store them in a storage device disposed inside the rehabilitation facility apparatus 30. Alternatively, the training apparatus 20 may transmit the physical ability information and the like to the presentation system 10 through the network N in response to an operation performed by the training staff or the like, upon detection of detection data, at predetermined intervals, or in response to an acquisition request from the presentation system 10.

The rehabilitation facility apparatus 30 is an information processing apparatus installed in a facility where the trainee 90 performs rehabilitation by using the training apparatus 20, such as a medical institution, or installed in or at least operated by a facility where the rehabilitation is managed. The rehabilitation facility apparatus 30 is a database system that manages at least the physical ability information and the like.

For example, the rehabilitation facility apparatus 30 receives the physical ability information and the trainee attribute information which have been acquired from the training apparatus 20 through the network N. Then, the rehabilitation facility apparatus 30 associates the received physical ability information with the trainee attribute information and stores them in an internal storage device. Further, the rehabilitation facility apparatus 30 may acquire detection data from the training apparatus 20 through the network N, and acquire the index data, the trainee attribute information, and the like by using an electronic medical record system (not shown) in a medical institution or the like, or by having a training staff enter such data. Alternatively, when the trainee 90 performs training by using an apparatus or the like other than the training apparatus 20, the rehabilitation facility apparatus 30 may acquire data corresponding to the above-described detection data by having a training staff enter the data.

Further, the rehabilitation facility apparatus 30 may also transmit the physical ability information and the like to the presentation system 10 through the network N in response to an operation performed by the training staff or the like, upon acquisition of the physical ability information or the like, at predetermined intervals, or in response to an acquisition request from the presentation system 10. Note that since the configuration of the rehabilitation facility apparatus 30 can be implemented by a publicly-known information system or the like, its detailed description is omitted.

The trainee terminal 40 is an information processing apparatus operated by a user on the trainee side. Examples of the user include a relative, a guardian, and the like of the trainee 90 as well as the trainee 90 himself/herself. The trainee terminal 40 has a function of performing communication through the network N, and is, for example, a personal computer or a portable information terminal such as a tablet terminal, a smartphone, and the like. The trainee terminal 40 receives a presentation request for the housing environment (i.e., a request for presenting the housing environment) through an input device according to an operation performed by a user on the trainee side, and transmits the received presentation request to the presentation system 10 through the network N. Further, the trainee terminal 40 receives presentation information of the housing environment from the presentation system 10 through the network N, and outputs (e.g., displays) the received presentation information to an output device such as a screen.

The presentation system 10 presents housing environment based on the after-rehabilitation-training physical ability of a trainee. The presentation system 10 is, for example, a server apparatus. Further, it is assumed that in the presentation system 10, a web server, an application server, and a database server are executed on an OS (Operating System), and a web application for carrying out a housing environment presentation process according to this embodiment is performed on the application server. However, the software configuration of the presentation system 10 is not limited to the above-described example. Further, details of the configuration of the presentation system 10 will be described later.

The environment provider terminal 51 and the environment provider terminal 52 are information processing apparatuses owned by environment providers. The “environment provider” refers to a person who is capable of providing a preset housing environment to the trainee. For example, the environment provider may be a person who can renovate the trainee's house according to the physical ability of the trainee. The environment provider may also be a person capable of providing care or assistance to the trainee when he/she lives his/her everyday life in his/her house. Alternatively, the environment provider may be a person who provides insurance to the trainee when he/she lives his/her daily life in his/her house.

The environment provider terminal 51 and the environment provider terminal 52 have a function of performing communication through the network N, and are, for example, personal computers, tablet terminals, portable information terminals such as smartphones, and the like. The environment provider terminal 51 and the environment provider terminal 52 receive a presentation request or the like of the housing environment through an input apparatus in response to an operation of a user belonging to the environment provider. The environment provider terminal 51 and the environment provider terminal 52 can transmit various kinds of information about the housing environment to the presentation system 10 through the network N.

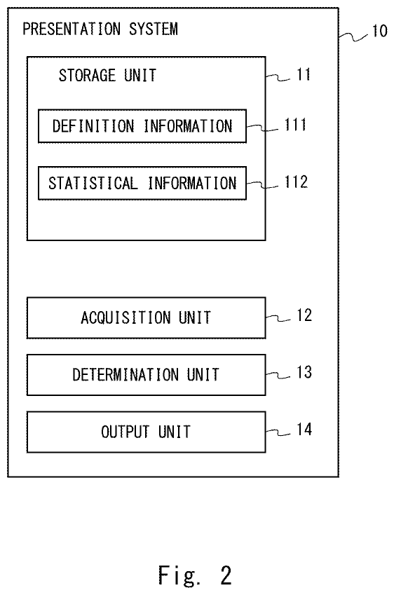

FIG. 2 is a block diagram showing a configuration of the presentation system 10 according to the first embodiment. The presentation system 10 includes a computer apparatus including at least an arithmetic circuit such as a CPU (Central Processing Unit) and a memory, and implements the functions described later by a combination of software and hardware. That is, the presentation system 10 includes a program for implementing the functions according to this embodiment. The presentation system 10 includes a storage unit 11, an acquisition unit 12, a determination unit 13, and an output unit 14 as its main components.

The storage unit 11 includes a non-volatile memory such as a flash memory, an EPROM (Erasable Programmable Read Only Memory), or an SSD (Solid State Drive). The storage unit 11 stores definition information 111 and statistical information 112.

The definition information 111 is information defining housing environment information according to the physical ability. That is, the definition information 111 according to this embodiment has a configuration in which the physical ability is associated with the housing environment. For example, the definition information 111 may be a conversion table or a conversion map defining a conversion rule from the physical ability information to the housing environment information. In this case, the definition information 111 may include a conversion table for each of the physical actions for a plurality of kinds of physical actions. Alternatively, the definition information 111 may be a converter, a function, a conversion algorithm, or a trained conversion model which converts information about the physical ability into housing environment information.

Note that the information about the physical ability includes a measured value(s) that is measured when the trainee 90 performs training by using the training apparatus 20 (i.e., includes a result of the training). The result of the training is, for example, the above-described detection data and/or sensor data detected by the training apparatus 20, and may be a set of measured values for a plurality of indexes. Further, the training is related to ADL and may be, for example, swallowing training related to meals, transfer training related to toilet actions, walking training, trunk balance training, etc.

Further, the information about the physical ability may include an index value of the physical ability of the trainee 90 during or after the training, or a physical evaluation value determined based on the result of the training. The physical evaluation value are, for example, the aforementioned Br. stage, the SIAS, the FIM, and the like. Therefore, the definition information 111 may be one in which the physical evaluation value is associated with the housing environment information.

Further, the information about the physical ability may be obtained by detecting and evaluating actions in daily life not defined as training by a sensor or the like. In this case, for example, information about the physical ability can be obtained by obtaining data from an acceleration sensor attached to a predetermined position of the trainee's body.

The housing environment information includes at least one of identification information of the housing environment, a type of a product or a service, a rank in the type, a specification of the housing environment, an installation condition, a detail of a construction work of the housing environment, and a detail of the service provided in the housing environment. The housing environment is an environment in which the trainee spends in his/her house after the training. Note that examples of the housing environment include so-called housing equipment for a renovation such as a handrail, a ramp, a wooden floor (for eliminating a difference in level on the floor) and a handrail in a bathroom, an outdoor passage, furniture, an electric appliance, and so on. The housing environment may also include care workers, caregivers, care insurance, home caregiver, transportation services, and the like.

The identification information of the housing environment, the product type, the rank in the type, and the specification of the housing environment may be acquired from, for example, a database for equipment managed by a renovation company. Note that the rank of the housing environment is information indicating a difference in the grade such as a price, a specification, or the like in housing equipment of the same type. That is, in the definition information 111, a different physical evaluation value may be associated with each of a plurality of ranks in the housing environment of the same type. In this way, it is possible to not only present the housing environment such as a “ramp”, but also present the housing environment in an appropriate rank according to the physical evaluation value.

The installation condition is, for example, information such as a length, a height, an inclination, an angle, and an installation place (e.g., outside the front door or inside the front door) of the ramp. Alternatively, in the case where a wheelchair can move more easily by changing an arrangement of existing furniture, the installation condition may include information about the arrangement of existing furniture. The setting information is information indicating a detail of a setting of an electric appliance. For example, when the electric appliance is an HSR (Human Support Robot), the setting information is a detail of a setting for providing support suitable for the physical ability information of the trainee 90. Further, when the electric appliance is a smart speaker equipped with a camera, the setting information is a detail of a setting of its software. Further, when the electric appliance is a camera, the setting information is, for example, a detail of a setting such as a setting for turning on a tipping-over detection function. Examples of the detail of the construction work of the housing environment include a construction work for extending a bathroom and a detail of that construction work.

The statistical information 112 is data related to training performed by a plurality of other trainees. The statistical information 112 includes, for example, data in which the content of training performed by a predetermined trainee, a type of a tool used by the trainee in the training, and the change in or degree of recovery of the physical ability of the trainee as a result of the training performed. The change in the physical ability may be based on the physical evaluation value (Br. stage, SIAS, FIM, etc.) described above, or based on the action level described later.

The acquisition unit 12 includes an interface for externally acquiring predetermined information, and acquires first physical ability information and plan information of the trainee. The first physical ability information is information about the physical ability of the trainee who performs the training for the purpose of recovering the physical ability, and indicates the physical ability of the trainee before the trainee starts predetermined training. The plan information is a plan of the training performed by the trainee.

The determination unit 13 includes an arithmetic circuit such as a CPU (Central Processing Unit) or an MPU (Micro Processing Unit) and executes a predetermined program to perform information processing. Specifically, the determination unit 13 receives the first physical ability information and the plan information from the acquisition unit 12, and estimates second physical ability information, which is the physical ability of the trainee after the end of the plan, using the first physical ability information and the plan information. The second physical ability information is information indicating the physical ability of the trainee after the end of the plan. The determination unit 13 estimates the second physical ability information as a result of performing the training included in the plan information by the trainee concerning the first physical ability information in accordance with a preset algorithm. At this time, the determination unit 13 can estimate the second physical ability information by further utilizing the statistical information 112. By using the statistical information 112, the determination unit 13 can prevent or minimize the deterioration of the accuracy of the second physical ability information to be estimated.

When the determination unit 13 estimates the second physical ability information, it determines the housing environment corresponding to the second physical ability information from the estimated second physical ability information and the definition information. The determination unit 13 generates presentation information including the determined housing environment, and supplies the generated presentation information to the output unit 14.

The output unit 14 includes an interface for outputting information received from the determination unit 13 to the outside. The output unit 14 outputs the presentation information including the determined housing environment. At this time, for example, the output unit 14 outputs the presentation information to one of the rehabilitation facility apparatus 30 and the trainee terminal 40 or both the rehabilitation facility apparatus 30 and the trainee terminal 40 through the network N.

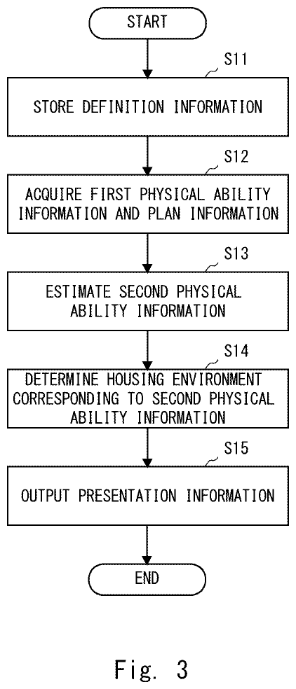

Next, processing performed by the presentation system 10 will be described with reference to FIG. 3. FIG. 3 is a flowchart of the presentation system according to the first embodiment. The flowchart shown in FIG. 3 is started, for example, by receiving an operation for starting a web application for carrying out a housing environment presentation process according to this embodiment.

First, the presentation system 10 stores the definition information 111 (Step S11). The presentation system 10 may acquire the definition information 111 by receiving the user's operation. The presentation system 10 may acquire the definition information 111 from the rehabilitation facility apparatus 30 or the like through the network N.

Next, the presentation system 10 acquires the first physical ability information and the plan information (Step S12). The presentation system 10 may acquire the first physical ability information and the plan information by receiving the user's operation. The presentation system 10 may also acquire the first physical ability information and the plan information from the rehabilitation facility apparatus 30 or the like through the network N.

Next, the determination unit 13 of the presentation system 10 estimates the second physical ability information (Step S13). Further, the determination unit 13 determines the housing environment corresponding to the estimated second physical ability information (Step S14), and supplies the presentation information including the determined housing environment to the output unit 14. Next, the output unit 14 of the presentation system 10 outputs the presentation information received from the determination unit 13 (Step S15).

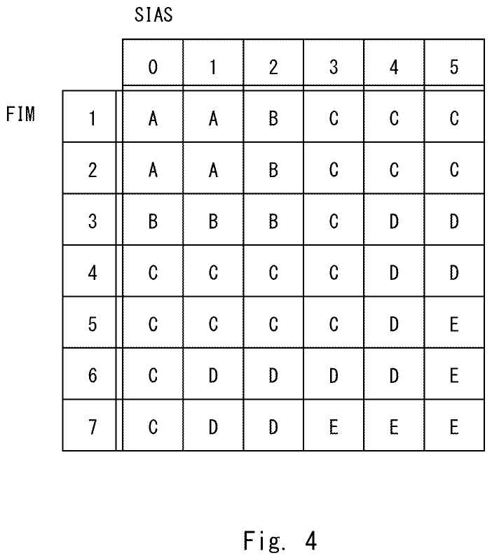

Next, a specific example of the definition information will be described. FIG. 4 shows a first example of the definition information according to the first embodiment. FIG. 4 is a conversion table as an example of the definition information 111 according to this embodiment. The conversion table shown in FIG. 4 is the definition information related to the physical ability of walking among physical abilities, and shows an example in which walking FIM and SIAS are employed.

In the conversion table of FIG. 4, the walking FIM is represented by values from 1 to 7 in the left column. Also, in the conversion table of FIG. 4, the SIAS of the foot-pat test is represented by values from 0 to 5 in the upper row. In the conversion table, the housing environment information corresponding to a combination of the values of the walking FIM and SIAS is shown in a matrix.

For example, when the walking FIM is 1 or 2, and the SIAS is 0 or 1, the housing environment information corresponds to a set A. The set A has been shown to provide the highest ranked housing environment for trainees with a low degree of independence of the walking FIM and a low SIAS. That is, for example, the set A includes wheelchairs, elevators, ramps, and the like. The set A also includes assistance services for dispatching a caregiver to assist the trainee.

For example, when the walking FIM is 3, and the SIAS is 0 to 2, or when the walking FIM is 1 to 3 and the SIAS is 2, the housing environment information corresponds to a set B. The set B corresponds to trainees whose degree of independence indicated by the walking FIM and SIAS is higher than that indicated by the set A. Thus, the housing environment information excludes elevators and includes ramps and handrails. In this case, the rank of the ramp may possibly decrease. As described above, the rank of the set of the housing environment information is set in the definition information 111 according to the degree of independence indicated by the combination of the walking FIM and the SIAS.

The conversion table of FIG. 4 described above shows the definition information related to a walking action. However, the definition information 111 may include the conversion tables corresponding to various physical actions. For example, exercise items that may be FIM include sub items such as self-care, excretion, a transfer (from one vehicle to another), and a transfer (from one place to another). Each of these sub items include a plurality of sub-sub items. The housing environment information concerning the conversion table includes corresponding equipment for each of the exercise items described above. For example, the conversion table for the toilet includes a handrail of a toilet room, a portable toilet, and a calling device. The conversion table related to the meal includes a meal providing service and a posture aiding tool. In this way, the presentation system 10 specify the housing environment more suitable for the physical ability information including various indexes using the definition information 111.

Note that in the definition information 111, a degree of rise in the physical evaluation value may be associated with the housing environment information. For example, the definition information 111 may include a definition of housing environment information by which a physical evaluation value, which is determined based on the result of the training of the trainee 90, may be raised.

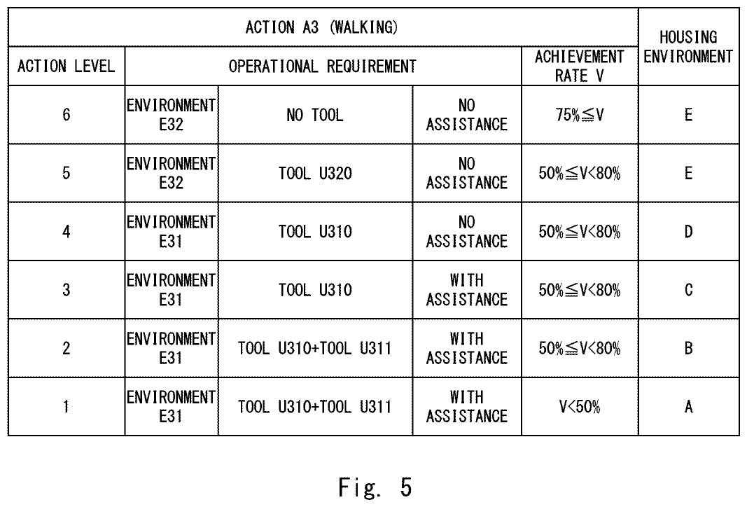

Next, a specific example of the definition information will be described with reference to FIG. 5. FIG. 5 shows a second example of the definition information according to the first embodiment. FIG. 5 is a conversion table as an example of the definition information 111 according to this embodiment. The conversion table shown in FIG. 5 shows an example in which the physical ability is defined by the action level and the defined action level is associated with the housing environment.

The action level is an index indicating the level of the physical action set using at least the information related to the kind of the tool used by the trainee in the physical action and an achievement degree of the physical action. The table of FIG. 5 shows the action levels related to an action A3 (walking) as an example of the action levels related to the physical action. The left column of the table of FIG. 5 shows six levels from 1 to 6 as the action levels of the action A3. The table of FIG. 5 shows operational requirements and achievement rates V when the action levels are 1 to 6. The operational requirements include the environment, the presence or absence of tools and assistance. A posture detection sensor may be attached to the trainee, so that the achievement rate V of the physical action can be determined from a signal of the attached posture detection sensor.

First, the action level shown in FIG. 5 will be described. For example, when the action level is 1, the trainee performs a predetermined action in an environment E31 while a tool U310 and a tool U311 are being used and a caregiver is assisting. In this case, if the achievement rate V of the action is less than 50%, the action level becomes 1. As shown in the second line from the bottom of FIG. 5, when the achievement rate of the action is 50% or higher and less than 80% with the above operational requirements, the action level becomes 2.

Further, as the operational requirements, when a predetermined action is performed in the environment E31 while the tool U310 is being used and the caregiver is assisting, if the achievement rate V of the movement is 50% or higher and less than 80%, the action level becomes 3. As the operational requirements, when a predetermined action is performed in the environment E31 while the tool U310 is being used and the caregiver is not assisting, if the achievement rate V of the action is 50% or higher and less than 80%, the action level becomes 4. Further, as the operational requirements, when a predetermined action is performed in an environment E32 while a tool U320 is being used and the caregiver is not assisting, if the achievement rate V of the action is 50% or higher and less than 80%, the action level becomes 5. As the operational requirements, in the environment E32, when a predetermined action is performed while the tool U320 is being used and the caregiver is not assisting, if the achievement rate V of the action is 75% or higher, the action level becomes 6.

The environment E31 and the environment E32 include, for example, indoor, outdoor, sloping land, and presence or absence of stairs and handrails. The above-described tool U310, tool U311, and tool U320 are, for example, an ankle-foot orthosis, a long-leg orthosis, a T-cane, and a quadruped cane.

Next, the housing environment shown in FIG. 5 will be described. The right column of the table in FIG. 5 shows the housing environment. The housing environment is represented by “A” to “E” corresponding to respective values of the action levels. For example, the set A indicating the housing environment corresponds to a case when the action level is 1. The set B corresponds to a case when the action level is 2. Similarly, the sets C, D, E and E correspond to cases when the action levels are 3, 4, 5 and 6, respectively.

Here, for example, the set A corresponding to the action level 1 is shown to provide the highest rank housing environment for the trainee with a low action level of walking. That is, for example, the set A includes wheelchairs, elevators, ramps, and the like. The set A also includes assistance services for dispatching a caregiver to assist the trainee.

For example, the set B corresponding to the action level 2 corresponds to a trainee whose action level of walking is relatively higher than the action level 1. Thus, the housing environment information excludes elevators and includes ramps and handrails. In this case, the rank of the ramp may possibly decrease. In this way, the rank of the set of the housing environment information is set in the definition information 111 according to the type of the action and the action level thereof.

The definition information 111 when the action level is employed has been described above. The action level is not based on subjective evaluation but is composed of objective data. By using such action levels, the presentation system 10 receives information about the action level of the trainee from the user. By doing so, the user can easily input information about the physical ability of the trainee.

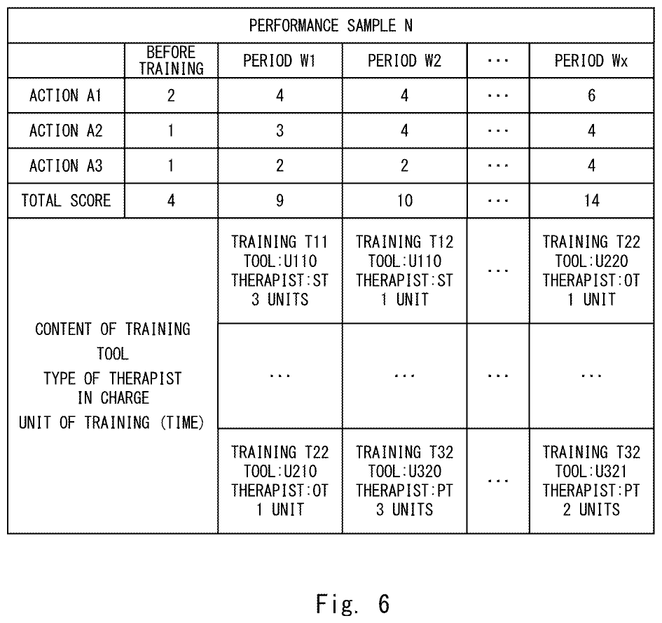

Next, the statistical information will be described with reference to FIG. 6. FIG. 6 shows the statistical information according to the first embodiment. The table of FIG. 6 shows an extracted performance sample N, which is the Nth sample included in the statistical information. The performance sample N is one sample data constituting the statistical information. That is, the statistical information includes a plurality of performance samples as shown in the table of FIG. 6. The performance sample N includes information in which the content of training performed by a predetermined trainee is associated with the degree of recovery of the physical action of the trainee as a result of the training performed.

The table in FIG. 6 shows the performance sample N in the upper row. In the table of FIG. 6, the transition of the action level is shown in the second row from the top. The transition of the action level is the action level as the performance of the trainee in the performance sample N. The action level is shown for each type of the physical action, separated by a predetermined period. Here, a period W1 is, for example, a period of one week as one unit. That is, the period W1 is the first week, a period W2 is the second week, and a period Wx is the xth week. The example shown in FIG. 6 shows the transitions of the action levels for three kinds of physical actions, an action A1, an action A2, and an action A3. The actions employed in the action A1, the action A2, and the action A3 are physical actions of the trainee in ADL, and correspond, for example, meal, toilet, transfer, and walking. That is, the example shown in FIG. 6 is information about the performance of the functional recovery training for the three kinds of physical actions of the action A1, the action A2, and the action A3. For convenience of explanation, a period W3 to a period Wx₋₁ and some columns of the details of the training are omitted. In the following similar tables, similar omissions may be made.

For example, the action level of the action A1 of the trainee before the training of the trainee of the performance sample N is 2. When the trainee performs the training, the action level of the action A1 becomes 4 after the end of the period W1, the action level of the action A1 is maintained at 4 after the end of the period W2, and the action level becomes 6 after the end of the period Wx. In addition, for example, the total score of the trainee of the performance sample N, which is the sum of the action A1, the action A2, and the action A3 before the training, is 4. By performing the training, the trainee has a total score of 9 after the end of the period W1, a total score of 10 after the end of the period W2, and a total score of 14 after the end of the period Wx.

The details of the training performed in each period are shown under the transition of the action level. The details of the training include the content of the training, the tool used in the training, the type of therapist in charge, and the unit of training (time). Here, the unit of the training indicates the time of training to be performed, and for example, one unit is defined as 30 minutes.

In the performance sample N shown in the table in FIG. 6, the total score of the trainee before the training is 4. The performance level after the end of the period W1 is 8. In the period W1, for example, training T11 and training T22 are performed. As shown in the table of FIG. 6, in the training T11 during the period W1, a tool U110 is used, a therapist in charge is ST (Speech Therapist), and the unit of training is three (which is 90 minutes). It is also shown in the table of FIG. 6 that in the training T22 performed during the period W1, a tool U210 is used, a therapist in charge is OT (Occupational Therapist), and the unit of training is one.

Likewise, the performance sample N shown in the table of FIG. 6 shows that the total score after the end of the period W2 is 10. According to the table of FIG. 6, training T12 and training T32 are performed during the period W2. In the training T12 during the period W2, the tool U110 is used, the therapist in charge is ST, and the unit of training is 1 unit. In the training T32 during the period W2, the tool U320 is used, and the therapist in charge is PT (Physical Therapist), and the unit of training is three. Further, according to the table of FIG. 6, training T22 and training T32 are performed during the period Wx. In the training T22 during the period Wx, the tool U220 is used, the therapist is charge is OT, and the unit of training is one. In the training T32 during period Wx, a tool U321 is used, the therapist is charge is PT, and the unit of training is two.

As described above, the performance sample includes the transition of the tool used, therapist in charge, or the action level of the physical action for the training performed by the predetermined trainee. It can also be said that the transition of the action level of the physical action shows the degree of recovery of the trainee. The determination unit 13 estimates the second physical ability information using the statistical information including a plurality of such performance samples.

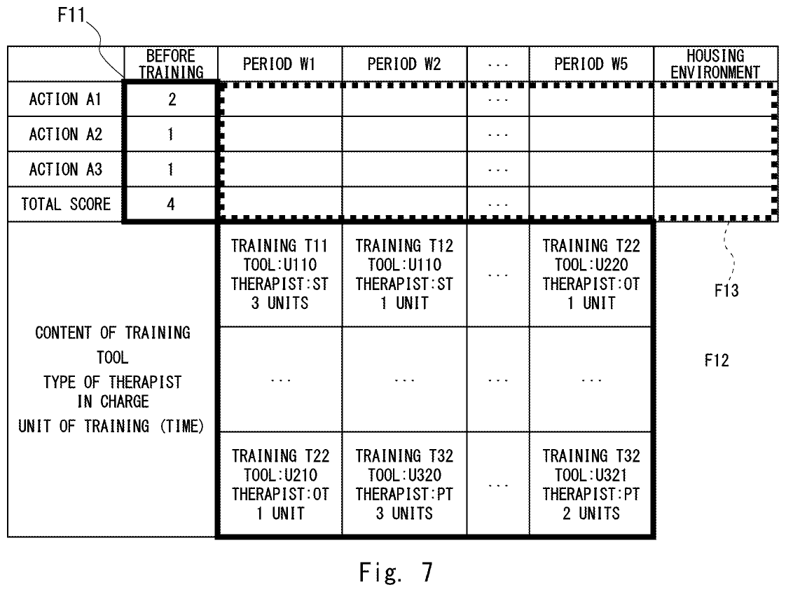

Next, information acquired by the acquisition unit 12 will be described with reference to FIG. 7. FIG. 7 shows an example of the information acquired by the acquisition unit according to the first embodiment. The table shown in FIG. 7 shows a relationship among the action level of the trainee, the content of the training performed by the trainee, and the housing environment information. The table of FIG. 7 has a first field F11, a second field F12, and a third field F13. The table of FIG. 7 shows that the acquisition unit 12 has acquired the first physical ability information and the plan information in the presentation system 10.

The first field F11 indicates the action level of the trainee before the training. That is, the information included in the first field F11 is the first physical ability information of the trainee before the start of the training. The first field F11 shown in FIG. 7 shows the action levels of the action A1, the action A2, and the action A3. The first field F11 also shows the total score obtained by summing the action levels of the action A1, the action A2, and the action A3. Specifically, according to the example shown in the table of FIG. 7, the action level of the action A1 of the trainee before the training is 2, and the action levels of the action A2 and the action A3 are both 1.

The second field F12 indicates the training plan performed by the trainee. The training plan includes, for example, the detail of training, the type of tool used in the training, the type of therapist in charge, and the unit of training. That is, the training plan includes items corresponding to the statistical information shown in FIG. 6. Thus, the determination unit 13 can estimate the second physical ability information using the information included in the second field F12.

The acquisition unit 12 can acquire the information shown in the first field F11 and the second field F12, for example, by the user inputting the information to the presentation system 10. Alternatively, the acquisition unit 12 may acquire the above information shown in the first field F11 and the second field F12, for example, from the rehabilitation facility apparatus 30 through the network N.

In the table of FIG. 7, the third field F13 shows the first physical ability information shown in the first field F11 and the information corresponding to the training plan shown in the second field F12. When the acquisition unit 12 acquires the first physical ability information and the plan information shown in FIG. 7, the determination unit 13 estimates the second physical ability information using the information acquired by the acquisition unit 12, and further determines the housing environment information corresponding to the estimated second physical ability information. That is, the determination unit 13 generates information corresponding to the third field F13. The table in FIG. 7 shows a state right after the acquisition unit 12 acquires the first physical ability information and the training plan. Thus, the third field F13 is empty.

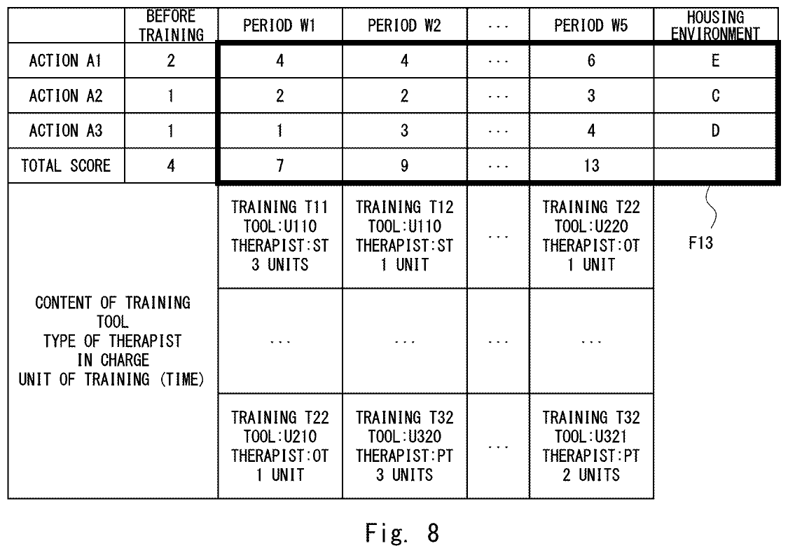

FIG. 8 shows an example of the presentation information according to the first embodiment. FIG. 8 shows a state in which the determination unit 13 generates the information corresponding to the third field F13 from the first physical ability information and the training plan acquired in FIG. 7. The determination unit 13 generates an estimated value of the action level corresponding to the period of the training. For example, the determination unit 13 estimates that the action level of the trainee at the end of the period W1 is 4 for the action A1, 2 for the action A2, and 1 for the action A3. The determination unit 13 estimates that the action level of the trainee at the end of the period W2 is 4 for the action A1, 2 for the action A2, and 3 for the action A3. Further, the determination unit 13 estimates that the action level of the trainee at the end of the period W5 is 6 for the action A1, 3 for the action A2, and 4 for the action A3.

Further, the determination unit 13 determines the housing environment corresponding to the estimated action level. The right column of the third field F13 is the housing environment information determined by the determination unit 13. For example, a case where the action A1 is the meal will be described. The housing environment corresponding to the action A1 after the end of the period W5 is a set E. Here, for example, the set E corresponding to the action level 6 of A1 has been shown to provide the lowest rank housing environment for a trainee whose action level of the meal is high. That is, for example, the set E includes, for example, a meal delivery service.

Although the first embodiment has been described above, the presentation system 10 according to the first embodiment is not limited to the above-described configuration. For example, in the presentation system 10, the acquisition unit 12 may also receive profile information of the trainee. An example of the profile information of the trainee is information indicating attributes of the trainee. The information indicating the attributes of the trainee is, for example, information indicating an age, a sex, a physique (a height, a weight, etc.), and a physical condition of the trainee. The environment provider terminals shown in FIG. 1 are an example for facilitating the description, and the number of providers of the housing environment is not limited, as long as it is one or more.

Another example of the trainee's profile information is symptom information of a disease(s) the trainee has suffered from. The symptom information can include a type(s) of a disease(s) (a name(s) of a disease(s) or a disorder(s)) as well as a location (damated part), such as stroke (cerebrovascular disorder), spinal cord injury, and, depending on the type, the classification of the disease or disorder. For example, strokes can be classified into cerebral infarction, intracranial hemorrhage (cerebral hemorrhage/subarachnoid hemorrhage), etc. Note that the symptom information may include information indicating an initial symptom of the above disease, a time when the symptom of the above disease appears, and a current symptom related to the above disease. The symptom information may further include symptoms that do not seem to be directly related to the rehabilitation, in addition to the information that can be considered that the trainee needs to perform rehabilitation mainly because of at least one of the symptoms included in the symptom information.

By acquiring such profile information of the trainee, the determination unit 13 of the presentation system 10 also uses the profile of the trainee to estimate the second physical ability information. By doing so, the presentation system 10 can improve the accuracy of the second physical ability information to be estimated.

The storage unit 11 may further store provider information related to a provider of the housing environment. The provider information may be supplied from the environment provider terminal 51 and the environment provider terminal 52 through the network N. In this case, the output unit 14 outputs the presentation information including the provider information related to the determined housing environment.

The training plan acquired by the acquisition unit 12 may be acquired by the presentation system 10 itself by generating the training plan from the first physical ability information and the statistical information instead of being externally input.

With the above-described configuration, the presentation system 10 can present a suitable housing environment according to the physical ability of the trainee. Therefore, according to the first embodiment, it is possible to provide a presentation system and the like which prevent or minimize an increase in the burden on the trainee.

### Second Embodiment

Next, a second embodiment will be described. The second embodiment is different from the first embodiment in that a storage unit according to the second embodiment further stores presentation information 113 and preparation information. The second embodiment differs from the first embodiment in that the presentation system according to the second embodiment issues a notification about a start of preparation of the housing environment corresponding to the preparation information.

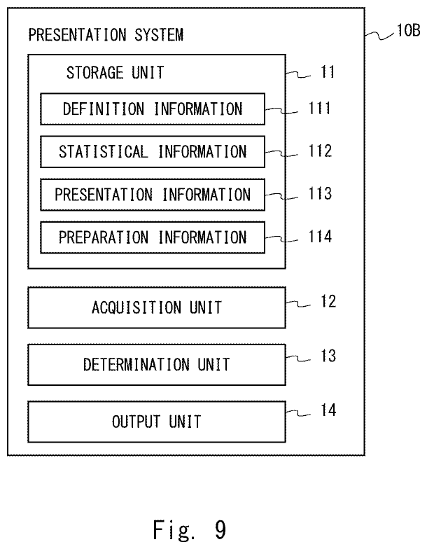

FIG. 9 is a block diagram of the presentation system according to the second embodiment. In a presentation system 10B shown in FIG. 9, the storage unit 11 stores the presentation information 113 and preparation information 114.

The presentation information 113 is generated when the determination unit 13 estimates the second physical ability information and then determines the housing environment information from the estimated second physical ability information. That is, the presentation system 10B outputs the presentation information and stores the presentation information related to the output in the storage unit 11. It can also be said that the presentation system 10B does not have the presentation information before the presentation information is generated. The presentation information 113 shown in FIG. 9 is information included in an area for storing the presentation information, and includes, for example, information indicating that the presentation information output by the presentation system 10B is not included.

The preparation information is information about a preparation period for preparing the housing environment. The preparation information relates to the housing environment information included in the definition information and includes the information about the preparation period necessary for providing each of the housing environment to the house of the trainee. For example, if the housing environment information includes a wheelchair, the preparation information includes a time required to arrange the wheelchair. For example, if the housing environment information includes a service assistant, the preparation information includes a preparation period for requesting the service assistant at an appropriate timing. For example, if the housing environment information includes a handrail, the preparation information includes a preparation period for renovating the house to add the handrail. In response to a request from the determination unit 13, the storage unit 11 supplies the preparation information 114 to the determination unit 13.

The determination unit 13 according to this embodiment estimates the second physical ability information, and after determining the housing environment information, determines to issue a notification related to the start of preparation of the housing environment at a time the preparation period before the end of the acquired training plan. The preparation period here is for preparing the housing environment included in the presentation information.

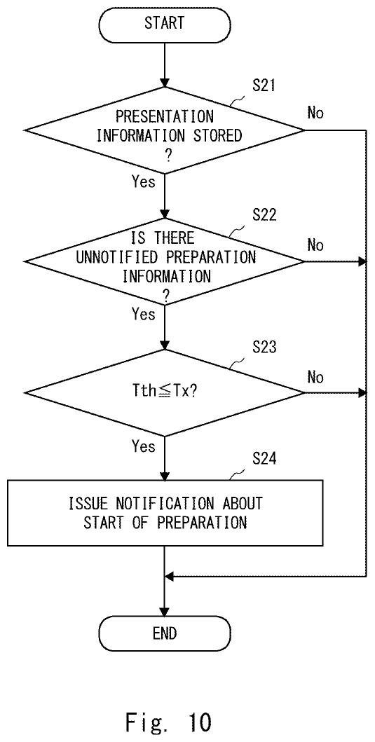

Next, the processing according to this embodiment will be described with reference to FIG. 10. FIG. 10 is a flowchart of the presentation system according to the second embodiment. The flowchart shown in FIG. 10 is set to start, for example, at a predetermined time every day in the presentation system 10B.

First, the presentation system 10B determines whether or not the presentation information is stored (Step S21). If the presentation system 10B does not store the presentation information (Step S21: No), the presentation system 10B ends the processing. On the other hand, if the presentation system 10B stores the presentation information (Step S21: Yes), the presentation system 10B proceeds to Step S22.

In Step S22, the presentation system 10B determines whether there is unnotified preparation information (Step S22). If it is not determined that there is unnotified preparation information (Step S22: No), the presentation system 10B ends the processing. On the other hand, if it is determined that there is unnotified preparation information (Step S22: Yes), the presentation system 10B proceeds to Step S23.

In Step S23, the presentation system 10B determines whether or not the unnotified preparation information has passed a notice period. For example, when the unnotified preparation period in the housing environment information is defined as Tth, and a period from the end of the training to the point at which this processing is being performed is defined as Tx, the presentation system 10B determines whether or not Tx is equal to or less than Tth (Step S23). If it is not determined that Tx is equal to or less than Tth (Step S23: No), the preparation information that has not been notified is present, but the timing of notification has not yet arrived. In this case, the presentation system 10B ends the processing.

On the other hand, if it is determined that Tx is equal to or less than Tth (Step S23: Yes), the preparation information that has not been notified is present, and the timing of notification has arrived. In this case, the presentation system 10B determines to issue the notification about the start of preparation (Step S24). When the notification related to the housing environment information is issued, the presentation system 10B ends the processing.

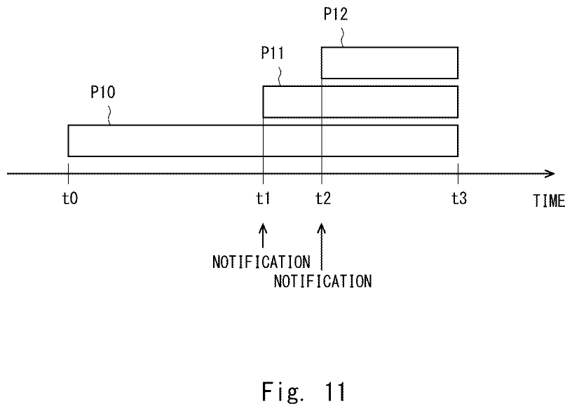

The processing of notifying the preparation information will be further described with reference to FIG. 11. FIG. 11 is a timing chart of the presentation system according to the second embodiment. In the timing chart shown in FIG. 11, the horizontal axis indicates time (or the passage of days). Each of the band-shaped rectangles shown above the horizontal axis indicates a period. A band-shaped period P10 shown for predetermined days t0 to t3 indicates a training period performed by the trainee. That is, the trainee performs the training during the period from the day t0 to the day t3, for example, in a rehabilitation facility. In other words, the trainee will start living in his/her house after the day t3. A period P11 and a period P12 shown above the period P10 indicate preparation periods of predetermined housing environment information.

Therefore, the determination unit 13 issues the notification indicating that it is a timing to start preparing the housing environment, which requires the period P11 as the preparation period, for example, on the day t1. The day t1 is the day that is the period P11 before the day t3 which is the day when the training ends. The period P11 is the preparation period of the housing environment included in the presentation information. Likewise, the determination unit 13 issues the notification indicating that it is a timing to start preparing the housing environment, which requires the period P12 as the preparation period, for example, on the day t2. The day t2 is the day that is the period P12 before the day t3. The period P12 is the preparation period of the housing environment included in the presentation information.

In the presentation system 10B described above, the storage unit 11 may store the presentation information, the acquisition unit 12 may acquire progress information regarding the training plan in a timely manner, and the determination unit 13 may update the second physical ability information according to the progress information. The determination unit 13 may also update the housing environment according to the updated second physical ability information. Thus, the presentation system can flexibly present the housing environment information.

In the presentation system 10B, the storage unit 11 may store the presentation information, and the determination unit 13 may update the second physical ability information according to the updated plan information and update the housing environment according to the updated second physical ability information when the plan information is updated. Thus, the presentation system can flexibly present the housing environment information.

The second embodiment has been described above. The presentation system 10B according to the second embodiment outputs the notification concerning the preparation information in a timely manner according to the progress of the training. Thus, according to the second embodiment, the housing environment can be prepared at a suitable timing taking into consideration of the timing when the training ends. Therefore, according to the second embodiment, it is possible to provide a presentation system and the like which prevent or minimize an increase in the burden on the trainee.

### Third Embodiment

Next, a third embodiment will be described. The third embodiment is different from the above-described embodiments in that a presentation system according to the third embodiment includes an instruction reception unit and a transmission unit.

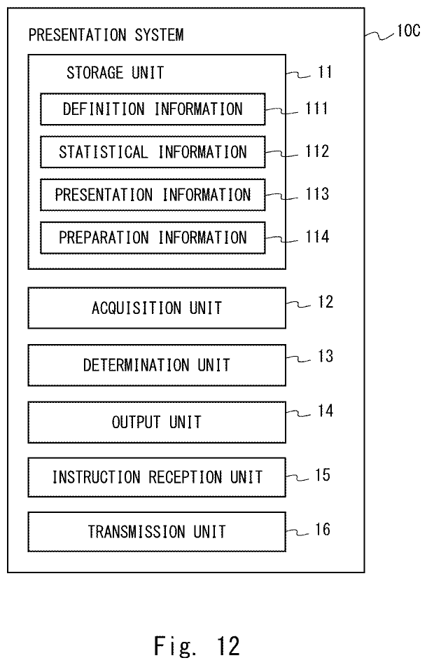

FIG. 12 is a block diagram of the presentation system according to the third embodiment. A presentation system 10C shown in FIG. 12 is different from the presentation system 10B according to the second embodiment in that the presentation system 10C includes an instruction reception unit 15 and a transmission unit 16.

The instruction reception unit 15 receives an instruction for selecting a provider for providing a housing environment. The instruction reception unit 15 receives such an instruction, for example, from the trainee terminal 40 through the network N. For example, after the output unit 14 outputs the presentation information, the instruction is received as an instruction for selecting a provider related to the output presentation information. The user can send a communication to the provider to start preparing the housing environment included in the housing environment information by giving such instruction. Alternatively, the selection information may be an instruction for ordering an arrangement of the housing environment.

The transmission unit 16 transmits selection information related to the selection to the provider concerning the selection received by the instruction reception unit 15. The transmission unit 16 can transmit the selection information to at least one of the environment provider terminal 51 and the environment provider terminal 52 through the network N. The provider who has received the selection information can recognize that he/she has been contacted regarding the field in which he/she is engaged.

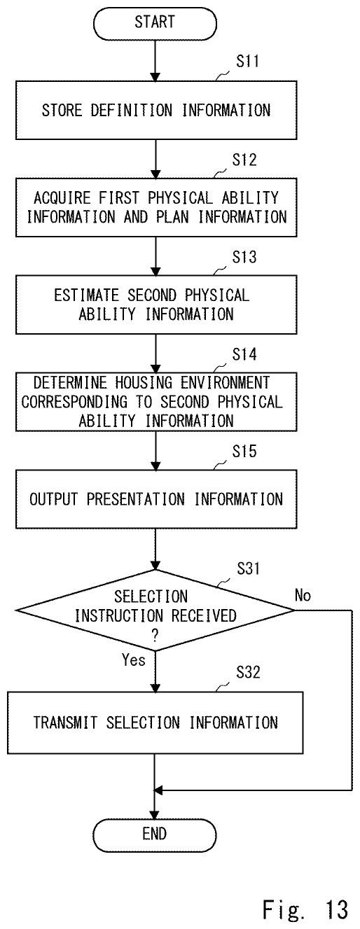

FIG. 13 is a flowchart of the presentation system according to the third embodiment. In the flowchart shown in FIG. 13, Steps S11 to S15 are the same as those of the flowchart shown in FIG. 3, and the processing after Step S15 is different from that of the flowchart shown in FIG. 3.

After outputting the presentation information in Step S15, the presentation system 10C determines whether or not a selection instruction is received from the user (Step S31). If it is not determined that the selection instruction is received from the user (Step S31: No), the presentation system ends the processing. On the other hand, if it is determined that the selection instruction is received from the user (Step S31: Yes), the presentation system transmits the selection information to the environment provider terminal 51 or the environment provider terminal 52 in response to the received instruction (Step S32).

The third embodiment has been described above. With such a configuration, the presentation system 10C can present the information for preparing the housing environment in a timely manner. Thus, by using the presentation system 10C, the trainee can smoothly arrange his/her life after ending the training. Therefore, according to the third embodiment, it is possible to provide a presentation system and the like which prevent or minimize an increase in the burden on the trainee.

Note that the present disclosure is not limited to the above-described embodiments, and may be modified as appropriate without departing from the spirit of the disclosure.

The program described above can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A presentation system comprising:
a storage unit configured to store definition information defining a housing environment according to a physical ability;
an acquisition unit configured to acquire first physical ability information of a trainee who performs training for the purpose of maintaining or recovering the physical ability before the training, and plan information which is a plan of the training;
a determination unit configured to estimate second physical ability information which is the physical ability of the trainee after an end of the plan based on the first physical ability information and the plan information, and determine the housing environment corresponding to the second physical ability information based on the definition information; and
an output unit configured to output presentation information including the determined housing environment.

2. The presentation system according to claim 1, wherein
the acquisition unit is further configured to acquire profile information of the trainee, and
the determination unit is configured to estimate the second physical ability information which is the physical ability of the trainee after the end of the plan based on the first physical ability information, the plan information, and the profile information.

3. The presentation system according to claim 1, wherein
the storage unit is further configured to store statistical information related to the training performed by a plurality of other trainees, and
the determination unit is configured to estimate the second physical ability information which is the physical ability of the trainee after the end of the plan based on the first physical ability information, the plan information, and the statistical information.

4. The presentation system of claim 1, wherein
the storage unit is configured to further store preparation information related to a preparation period for preparing the housing environment,
the determination unit is configured to determine that a notification concerning a start of preparation of the housing environment is to be issued at a time during the preparation period before the end of the plan, the preparation period being for preparing the housing environment included in the presentation information, and
the output unit is configured to output the notification.

5. The presentation system according to claim 1, wherein
the storage unit is configured to store the presentation information,
the acquisition unit is configured to acquire progress information regarding the training plan, and
the determination unit is configured to update the second physical ability information according to the progress information and update the housing environment according to the updated second physical ability information.

6. The presentation system according to claim 1, wherein
the storage unit is configured to store the presentation information, and
when the plan information is updated, the determination unit is configured to update the second physical ability information according to the updated plan information, and update the housing environment according to the updated second physical ability information.

7. The presentation system according to claim 1, wherein
the storage unit is further configured to store provider information related to a provider of the housing environment, and
the output unit is configured to output the presentation information including the provider information related to the determined housing environment.

8. The presentation system according to claim 7, further comprising:
an instruction reception unit configured to receive an instruction for selecting the provider; and
a transmission unit configured to transmit selection information related to the selection to the provider related to the selection received by the instruction reception unit.

9. A presentation method comprising:
storing definition information defining a housing environment according to a physical ability;
acquiring first physical ability information of a trainee who performs training for the purpose of maintaining or recovering the physical ability before the training, and plan information which is a plan of the training;
estimating second physical ability information which is the physical ability of the trainee after an end of the plan based on the first physical ability information and the plan information;
determining the housing environment corresponding to the second physical ability information based on the definition information; and
outputting presentation information including the determined housing environment.

10. A non-transitory computer readable medium storing a program that causes a computer to execute:
storing definition information defining a housing environment according to a physical ability;
acquiring first physical ability information of a trainee who performs training for the purpose of maintaining or recovering the physical ability before the training, and plan information which is a plan of the training;
estimating second physical ability information which is the physical ability of the trainee after an end of the plan based on the first physical ability information and the plan information;
determining the housing environment corresponding to the second physical ability information based on the definition information; and
outputting presentation information including the determined housing environment.
